(19) United States
(12) Patent Application Publication
(10) Pub. No.: US 2002/0177798 A1
(43) Pub. Date: Nov. 28, 2002

(54) Swab with olive oil

(76) Inventor: Kenneth Davidson, Dayton, OH (US)

Correspondence Address: William M. Selenke, 28 DeWitt Street, Cincinnati, OH, 45218, US

(21) Appl. No.: 10/121,755

(22) Filed: Apr. 12, 2002

Related U.S. Application Data
(60) Provisional application No. 60/283,940, filed on Apr. 16, 2001.

(51) Int. Cl. A61M 35/00; A61F 13/00

(52) U.S. Cl. 604/3; 604/304

(57) ABSTRACT

Swabs with adsorbed olive oil are manufactured in sealed containers or flexible packages. The swab can be either swabs on the end of sticks or plain flat absorbent gauze. The use of these olive oil swabs includes heating the sealed container or package in water, opening that container or package, removing the swab, and applying the olive oil to the afflicted area.

### FIELD OF INVENTION

The invention is concerned with the manufacture of swabs and bandages impregnated with olive oil and the use of olive oil as an unguent for inflamed and injured tissues, especially ears and noses.

### BACKGROUND

The medicinal properties of olive oil in wound dressings have been known since antiquity. “He went to him and bandaged his wounds pouring on oil and wine.” Luke 10:34. An example of a modem type “Q-tip”R swab is Deford U.S. Pat. No. 2,006,539. Examples of a modern type “Q-tip”R swabs adapted to apply mendicants are U.S. Pat. No. 2,490,168, Strauss and U.S. Pat. No. 3,958,571 Bennington. Placing absorbent tips of bandage materials on a stick is a long established patent and commercial art.

Olive oil, often called “sweet oil,” is a common, accepted home remedy for earache. The sweet oil is warmed and placed in the ear. The warmth and soothing effect of the oil does relieve earache. Likewise, olive oil is used an ointment for small dried areas of the skin and small irritated or scraped areas of the skin. Various oils are used with pads to remove makeup. Likewise, olive oil on stick swabs can be comforting to constrution workers in a dusty environment to clean out their nose. However, neither in the patent or commercial literature are references to swabs presoaked with sweet oil. A medicinal first aid pad manufactured with olive oil placed in the absorbent part of such pads would have certain advantages. They would be less messy and more convenient then bottles of olive oil. A second advantage is that no bacteria or other infectious agents in would be present in the swab with sterilized sweet oil after the combination is sterilized with common sterilization techniques.

Likewise, the medicinal first aid pad of present invention is concerned with the process of application of olive oil to affected areas. This process includes heating the pad or swab by placing the tube container or packet in warm water; and after the pad or swab is warmed, removing it from the water, opening the package or pad, and applying the olive oil to the afflicted site.

### BRIEF DESCRIPTION OF FIGURES

FIG. 1 show a standard single swab in a container tube with a reservoir of olive oil in the bottom.

FIG. 2 shows an olive oil saturated swab in a partially opened flexible package.

### DETAILED DESCRIPTION OF FIGURES

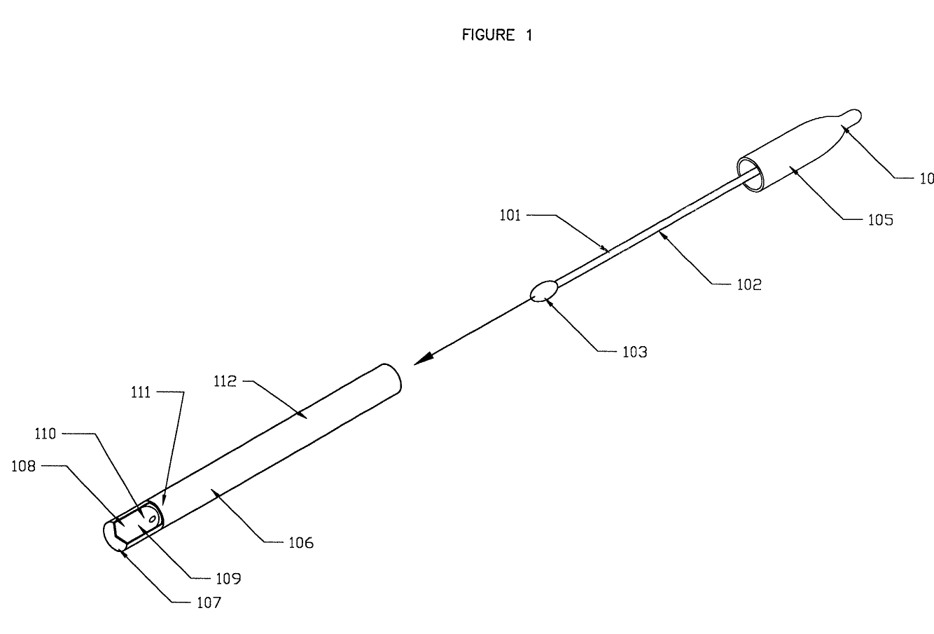

FIG. 1 shows a swab 101. Swab 101 has stick part 102 and an absorbent cotton or cotton-like pad 103 placed on the first end on stick part 102. The second end 104 of stick part 102 is placed in cover sleeve 105. Cover sleeve 105 covers and seals container tube 106. which has an inner area 112 and a terminus 107, which terminus 107 creates a lower part 108 of container tube 106. Optionally, the olive oil 109 can be placed in an absorbent wad 110 in lower part 108. Olive oil 109 is isolated from the inner area 112 of container tube 106. A readily penetrable barrier 111 allows ready access to olive oil 109. That is to note that olive oil can be placed during manufacture either in the absorbent wad 110 in lower part 108, or on the absorbent cotton or cotton-like pad 103 placed on the first end on stick part 102. In fact, olive oil could be placed on both absorbent wad 110 and first end pad 103 on stick part 102.

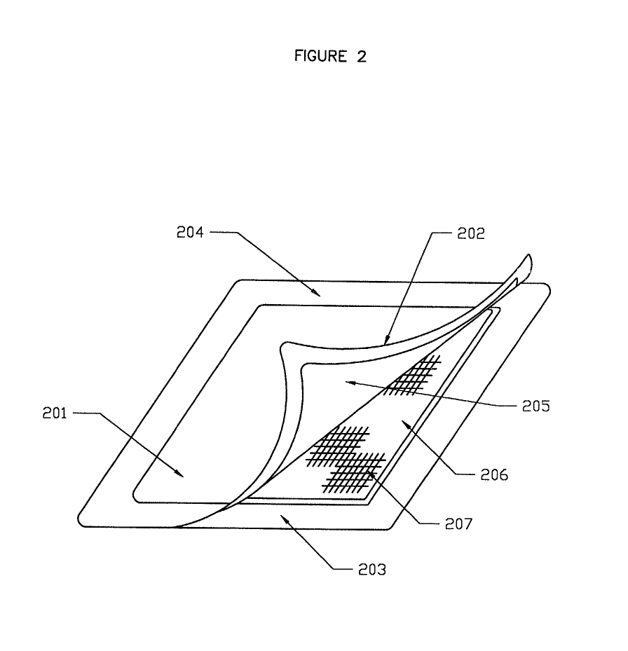

FIG. 2 shows a sealed packet 201, Packet 201 has a front side 202 and a back side 203. During manufacture front side 202 and back side 203 are sealed via leal 204. In the compartment 205 is placed a pad of absorbent material such as cotton gauze 206. Cotton gauze 206 is saturated with olive oil 207. This sealed packet 201 is show partially opened to access the gauze 206 saturated with olive oil 207.

I claim:

1) A medicinal first aid pad comprising:
a stick part with a first end and a second end;
an absorbent cotton or cotton-like pad placed on said first end on said stick part;
said second end is placed in a cover sleeve;
said cover sleeve covers and seals a container tube;
said container tube has a terminus which creates a lower part of said container tube;
olive oil is placed in an absorbent wad in said lower part;
said olive oil in said lower part is isolated from the inner area of said cover sleeve by a readily penetrable barrier;
said barrier allows ready access to said olive oil

2) A medicinal first aid pad comprising:
a stick part with a first end and a second end;
an absorbent cotton or cotton-like pad placed on said first end on said stick part;
said second end is placed in a cover sleeve;
said cover sleeve covers and seals a container tube;
olive oil is absorbed on said absorbent cotton or cotton-like pad placed on said first end of said stick part.

3) A medicinal first aid pad comprising;
a sealed packet with a front side and a back side;
said sealed packet contains a pad of absorbent material such as cotton gauze;
said absorbent material contains olive oil.

4) A process of applying olive oil to affected areas comprising the steps of:
heating a container or packet by placing said container or packet in warm water
said container or packet contains a swab
said swab has absorbed olive oil;
and after said container or packet is warmed;
removing said container or packet from the water;
opening said container or packet;
and applying said absorbed olive oil on said swab to the afflicted site.
